(19) United States
(12) Patent Application Publication
(10) Pub. No.: US 2014/0017343 A1
(43) Pub. Date: Jan. 16, 2014

(54) METHOD FOR MANUFACTURING DRUGS THAT ENHANCE BRAIN FUNCTION

(71) Applicant: CHE-HAO LIN, NEW TAIPEI CITY (TW)

(72) Inventor: CHE-HAO LIN, NEW TAIPEI CITY (TW)

(73) Assignee: SUN PLAZA INTERNATIONAL CO., LTD., NEW TAIPEI CITY (TW)

(21) Appl. No.: 13/616,837

(22) Filed: Sep. 14, 2012

(30) Foreign Application Priority Data
Jul. 13, 2012 (TW) 101125285

(51) Int. Cl. A61K 36/185; A61P 25/28; A61P 25/00

(52) U.S. Cl. 424/725

(57) ABSTRACT

A method for manufacturing drugs that enhance brain function, includes the steps of: dip a predetermined amount of Ludwigia octovalvis in an extraction solution to extract a Ludwigia octovalvis extraction solution and a Ludwigia octovalvis residue; separate the Ludwigia octovalvis residue from the first Ludwigia octovalvis extraction solution; and conduct a drying process to the Ludwigia octovalvis extraction solution to obtain an extracted Ludwigia octovalvis substance.

This application claims the benefits of the Taiwan Patent Application Serial NO. 101125285 filed on Jul. 13, 2012, the subject matter of which is incorporated herein by reference.

### BACKGROUND OF THE INVENTION

1. Field of the Invention

The present invention relates to a method for manufacturing drugs, more particularly to a method for manufacturing drugs, in which Ludwigia octovalvis is used as raw material and which enhance brain function and delay neurodegradation.

2. Description of the Prior Art

As global population tends to get older in age, a majority of the population suffers from Alzheimer's disease such that several countries are attending to this problem with a great concern. According to a research, an estimation of approximately more than 24 millions people suffers from this disease all over the world. In case a patient suffering from this disease gets worse, he or she needs the help from nearby persons in all aspects of life, such as bathing, eating, going to the toilet and so on. Since the patient with Alzheimer's disease needs to be taken care of day in and day out, family members and the patient's relatives and friends are often encountered with a great deal of inconveniences and discomfort in their daily lives.

So far, there is still no accurate diagnosis and effective treatment for Alzheimer's disease, and therefore the scientists all over are working hard and conducting researches in order to develop a new drug for treating this disease.

Therefore, the inventor of the present invention believes that it is urgently needed to develop a new method for manufacturing drugs that can effectively boost or enhance brain function and delay neurodegradation in people, more particularly in the elder people.

### SUMMARY OF THE INVENTION

As above mentioned, in the prior art technique, there is no clear or effective method of treating Alzheimer's disease, and in order to find other treatments or drugs, the inventor of the present application has recently developed a drug to enhance brain function and delay neurodegradation method for producing the drugs.

An experiment conducted according to the present invention provides a method for manufacturing drugs to enhance brain function and delay neurodegeneration, the drugs or medicines accordingly produced can improve brain function, and can delay neurodegeneration.

The method of the present invention for manufacturing drugs that enhance brain function, wherein Ludwigia octovalvis is used as raw material, the method includes the steps of: dip a predetermined amount of Ludwigia octovalvis in a first extraction solution to extract a first Ludwigia octovalvis extraction solution and a Ludwigia octovalvis residue; separate the Ludwigia octovalvis residue from the first Ludwigia octovalvis extraction solution; and conduct a drying process to the first Ludwigia octovalvis extraction solution to obtain an extracted Ludwigia octovalvis substance.

Preferably, after the drying process, the method of the present invention further includes a step, where the extracted Ludwigia octovalvis substance is redissolved in the first extraction solution in such a manner that the extracted Ludwigia octovalvis substance has a concentration ranging between 0.0005% ˜0.5%.

In the manufacturing method of the present invention, the predetermined amount of Ludwigia octovalvis is dipped in the first extraction solution for 4˜12 hours. Preferably, the predetermined amount of Ludwigia octovalvis is dipped in the first extraction solution at a low temperature ranging 2˜10° C. In addition, after the predetermined amount of Ludwigia octovalvis is dipped in the first extraction solution for 4˜12 hours, and then first the extraction solution is heated for 10˜60 minutes.

In the manufacturing method of the present invention further includes a substep, where the Ludwigia octovalvis residue is dipped in a second extraction solution, after which the second extraction solution is heated and filtered in such a manner to extract a second Ludwigia octovalvis extraction solution, which, in turn, is combined with the first Ludwigia octovalvis extraction solution.

Preferably, the manufacturing method of the present invention further includes a step, wherein the predetermined amount of Ludwigia octovalvis is ground prior to dipping (the same) in the first extraction solution.

The manufacturing method of the present invention further includes a step, wherein separating the Ludwigia octovalvis residue from the first Ludwigia octovalvis extraction solution is conducted via centrifugal filtering means.

Preferably, the manufacturing method of the present invention further includes in one step, wherein after the predetermined amount of Ludwigia octovalvis is dipped in the first extraction solution, the first extraction solution is heated at a temperature ranging from 50˜100° C. In addition, the drying process in one of the step is a freeze drying process.

In the manufacturing method of the present invention an alcohol solution serves as the first extraction solution. Preferably, after dipping the predetermined amount of Ludwigia octovalvis in the first extraction solution, the first extraction solution is heated indirectly through a water bath up to a temperature ranging from 50˜100° C. Moreover, the drying process in one of the step is a decompress concentration process.

The advantages provided by the manufacturing method of the present invention are as follows.

From above mentioned paragraphs, it is obvious that so far no Ludwigia octovalvis is ever used as raw material for manufacturing the drugs that enhance brain function and that delay neurodegradation.

In addition, since the Ludwigia octovalvis serves as the raw material and since the same is easily found in botanical plants, the manufacturing cost can be reduced to manufacture the drugs with the manufacturing method of the present invention.

### BRIEF DESCRIPTION OF THE DRAWINGS

Other features and advantages of this invention will become more apparent in the following detailed description of the preferred embodiments of this invention, with reference to the accompanying drawings, in which:

FIG. 1 shows a block diagram illustrating the steps in a method of the present invention for manufacturing drugs that can boost or enhance brain function and delay neurodegeneration;

FIG. 2 shows a graph of an experiment carried out in the present invention illustrating the concentration of an extracted Ludwigia octovalvis substance relative to survival rate of male fruit flies (Drosophila melanogaster);

FIG. 3 shows a graph of an experiment carried out in the present invention illustrating the concentration of an extracted Ludwigia octovalvis substance relative to survival rate of female fruit flies;

FIG. 4 shows a graph of an experiment carried out in the present invention illustrating the concentration of an extracted Ludwigia octovalvis substance relative to learning ability (short-term memory) in fruit flies;

FIG. 5 shows a graph of an experiment of present invention carried out using novel object recognition task and illustrating the concentration of an extracted Ludwigia octovalvis substance relative to object recognition index in mice; and

FIG. 6 shows a graph of an experiment of the present invention carried out within an elevated T-maze and illustrating the concentration of the extracted Ludwigia octovalvis substance relative to the memory ability in mice.

### DETAILED DESCRIPTION OF THE PREFERRED EMBODIMENTS

The method of the present invention for manufacturing drugs, which enhance brain function and delay neurodegradation and in which Ludwigia octovalvis is used as raw material. Since the manufacturing steps differ from one another, the following paragraphs only illustrate an example for better understand of the present invention; however the scope of the present invention should not be limited to those given in the examples.

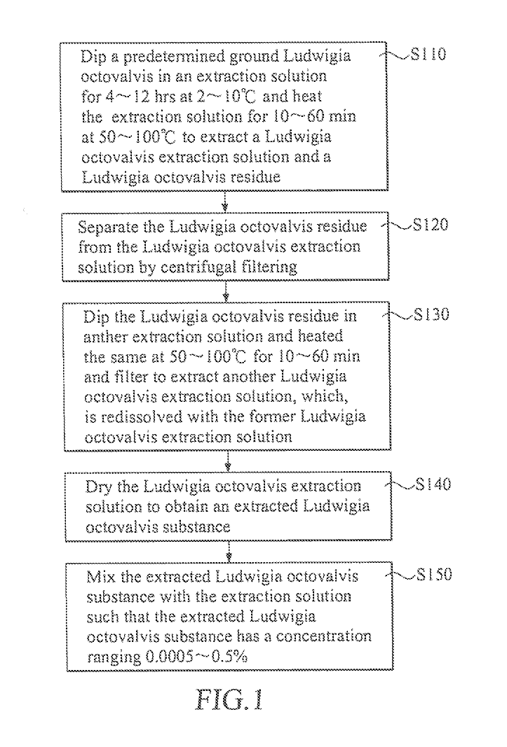

FIG. 1 shows a block diagram illustrating the steps in a method of the present invention for manufacturing drugs that can boost or enhance brain function and delay neural degeneration in people, especially in elder people. As illustrated, Ludwigia octovalvis is used as raw material and the method for manufacturing drugs according to the present invention includes steps of: in accordance with the step S110, a predetermined amount of Ludwigia octovalvis is ground, after which, the ground predetermined Ludwigia octovalvis is dipped in a first extraction solution at a low temperature ranging 2° C.˜10° C. for 4˜12 hours. Then the first extraction solution is heated at the temperature ranges 50° C.˜100° C. for 10˜60 minutes in order to extract a first Ludwigia octovalvis extraction solution and a Ludwigia octovalvis residue.

In this embodiment, since the predetermined amount of Ludwigia octovalvis is ground first of all, and then is dipped in the first extraction solution so that extraction work relative to the first Ludwigia octovalvis extraction solution is effective and the extraction time is shortened. The first extraction solution is selected from a nontoxic but edible and fine stable solutions, such as a water-solvent solution and an alcohol solution, such that during the extraction process of the first Ludwigia octovalvis extraction solution and the extraction of the Ludwigia octovalvis residue, there is no chemical reactions and the extraction can he carried out in a stable manner. In addition, when the alcohol solution is chosen as the first extraction solution, the first extraction solution is heated indirectly through a water bath.

In the step S120, centrifugal filtering means is conducted in order to separate the Ludwigia octovalvis residue from the first Ludwigia octovalvis extraction solution.

Afterwards in the step S130, the Ludwigia octovalvis residue (separated from the first Ludwigia octovalvis extraction solution) is dipped in a second extraction solution, after which the second extraction solution is heated at the temperature ranging 50° C.˜100° C. for 10˜60 minutes and centrifugal filtering means is conducted in order to filter in such a manner to extract a second Ludwigia octovalvis extraction solution, which, in turn, is combined with the first Ludwigia octovalvis extraction solution.

In the step S140, conduct a drying process to the first Ludwigia octovalvis extraction solution so as to obtain an extracted Ludwigia octovalvis substance. In the event, the first extracted solution is water solvent, a freeze drying process is conducted in order to obtain the extracted Ludwigia octovalvis substance. In the event, the alcohol solution is chosen as the first extracted solution is, a decompress concentration process is conducted to the first Ludwigia octovalvis extraction solution so as to obtain the extracted Ludwigia octovalvis substance.

Finally in the step S150, the extracted Ludwigia octovalvis substance is redissolved in the first Ludwigia octovalvis extraction solution in such a manner that the extracted Ludwigia octovalvis substance has a concentration ranging between 0.0005%˜0.5%.

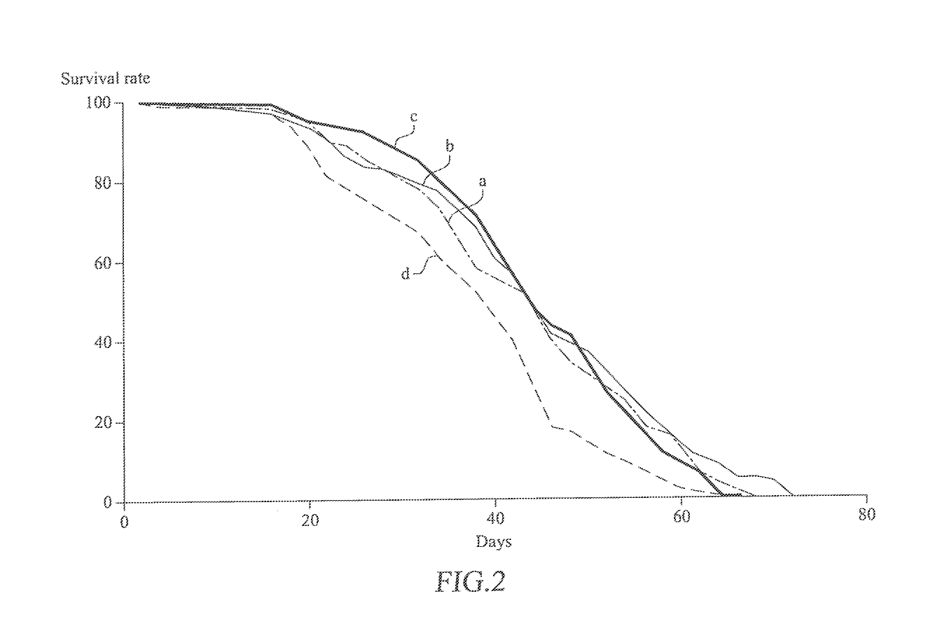

FIG. 2 shows a graph of an experiment carried out in the present invention illustrating the concentration of the extracted Ludwigia octovalvis substance relative to survival rate of male fruit flies. The graph represents the result of the experiment carried out under a temperature of 25° C., where the extracted Ludwigia octovalvis substance containing different concentration is fed to the male fruit flies, which are divided into (a), (b), (c) and (d) groups and are disposed in different 4 cages, then observe survival rate changes. In this experiment, the environmental condition is provided with 12/12 hours on/off light cycle and the humidity of the environment is maintained between 50˜65%.

Firstly, the (a) group is fed on edible food containing the extracted Ludwigia octovalvis substance with concentration of 0.005%; the (b) group is fed on edible food containing the extracted Ludwigia octovalvis substance with concentration of 0.01%; the (c) group is fed on edible food containing the extracted Ludwigia octovalvis substance with concentration of 0.1% while the (d) group is fed on edible food without containing the extracted Ludwigia octovalvis substance. In this experiment, the edible food contains 2˜20% sugar, 2˜20% yeast powder and 0.5˜2% agar and different concentration of the extracted Ludwigia octovalvis substance.

The survival rate of the (a), (b), (c) and (d) groups is recorded after every two days. As shown in the graph, fruit flies belonging to the groups (a), (b) and (c) survive longer when compared the flies belonging to the group (d). Hence, this experiment, proves that those fruit flies fed on edible food containing the extracted Ludwigia octovalvis substance have high survival rate (longer lifespan).

It is noted that , from the 20^{th} day to the 40^{th} day, the male flies belonging to the group (c) (concentration of 0.1%) have a higher survival rate, and after the 40^{th} day onward, the male flies belonging to the group) (a) (concentration of 0.005%) and the group (b) (concentration of 0.01%) respectively have higher survival rate. In short, though the male flies belonging to the group (b) from the 20^{th} day to the 40^{th} day have a lower survival rate than the group (c), the male flies of the group (b) have a higher survival rate after the 40^{th} day onward when compared to the group (e).

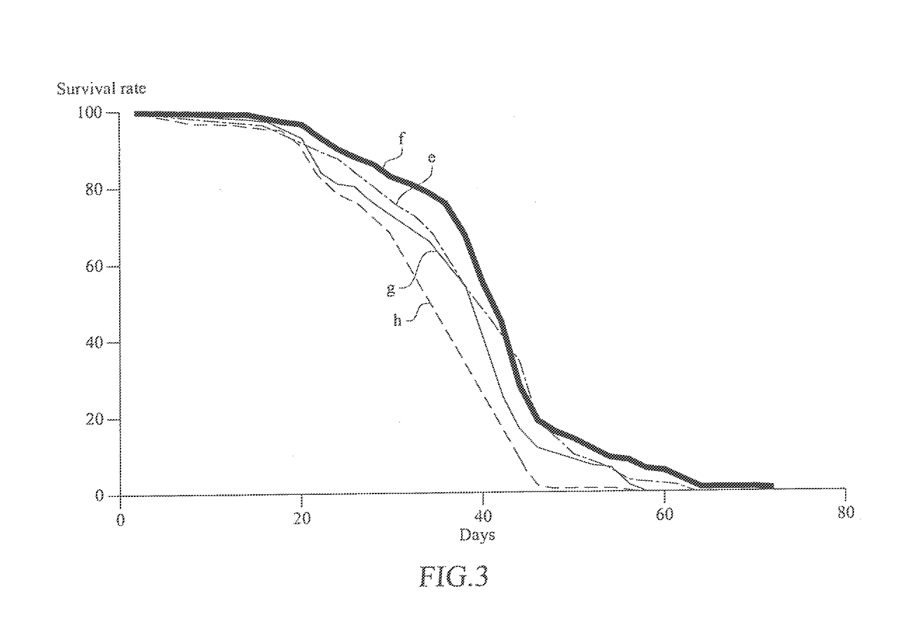

FIG. 3 shows a graph of an experiment carried out in the present invention illustrating the concentration of an extracted Ludwigia octovalvis substance relative to survival rate of female fruit flies. As shown, this is the survival result of the experiment which is similar the above survival experiment, the difference resides in that the above studies are conducted on the male fruit flies while the present experimental object is based on female fruit flies. The female fruit flies are divided into four groups (e), (f), (g), and (h) respectively. It is found in the graph that the female fruit flies of the groups (e), (f), (g) respectively have higher survival rate than the female fruit flies of the group (h). Note in this experiment, the female fruit flies of group (f) (concentration 0.01%) have the highest survival rate when compared to the remaining groups throughout the experiment.

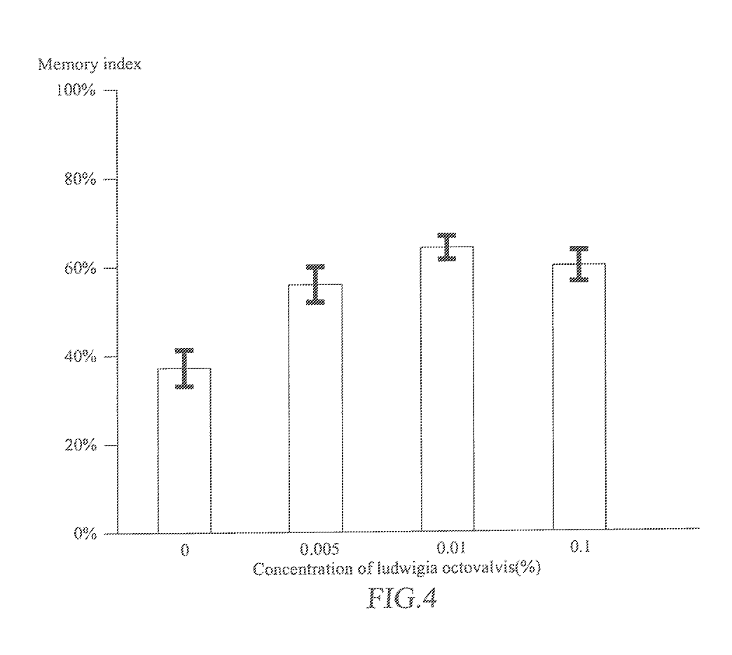

FIG. 4 shows a graph of an experiment carried out in the present invention illustrating the concentration of an extracted Ludwigia octovalvis substance relative to learning ability (short-term memory) in fruit flies. As illustrated, this experiment is generally called “Pavlovian Olfactory Associative Learning” in which odors and electroshocks are applied to fruit flies to monitor the learning and memory performance. Based on this test, one can judge the quality of learning and memory in fruit flies.

To conduct the “Pavlovian Olfactory Associative Learning” test, fruit flies are maintained under an environment of 25° C. and are fed on edible food containing 0%, 0.005%, 0.01% and 0.1% of the extracted Ludwigia octovalvis substance. The test is then carried out at 20 days of age.

In this test, two different odors; i.e., 3-OCT (3-Octanol) and 4-MCH (4 -Methylcyclohexanol), which respectively have a ratio of 1.5:1000 and 1:1000 in mineral oil. Both 3-OCT and 4-MCH odors are disliked by fruit flies. The whole test is conducted in the darkroom.

The first step is exposing the fruit flies to 3-OCT for 60 seconds, during which, an electroshock (strength for 70 volts) is applied for 1.5 seconds and 3.5 seconds of rest in an alternate manner. Immediately after 45 seconds of resting, the fruit flies are subsequently exposed to 4-MCH for 60 seconds without application of electroshock (3-OCT and 4-MCH can be exchanged alternately). The fruit flies are then allowed to rest for 30 seconds. Finally, the fruit flies are allowed to enter a T-maze with 3-OCT and 4-MCH provided at two opposing arms of the T-maze for a period of two minutes. The performance index or memory index is calculated by subtracting the number of the fruit flies entering the 3-OCT arm from those entering the 4-MCH arm, and then dividing by the total number of the fruit flies and is multiplied by 100%.

In the second step of the test, the odor paired with electroshock are exchanged and the test is carried out as described above. The average memory index is obtained by balancing the two results.

In FIG. 4, fruit flies fed on edible food, containing 0% of the extracted Ludwigia octovalvis substance has a memory index less than 40%; fruit flies fed on edible food containing 0.005% of the extracted Ludwigia octovalvis substance have memory index around 60%; fruit flies fed on edible food containing 0.01% and 0.1% of the extracted Ludwigia octovalvis substance have memory index exceeding over 60%. From this result, it is noticeable that the feeding food containing the extracted Ludwigia octovalvis substance can significantly enhance learning performance (short-term memory) in fruit flies.

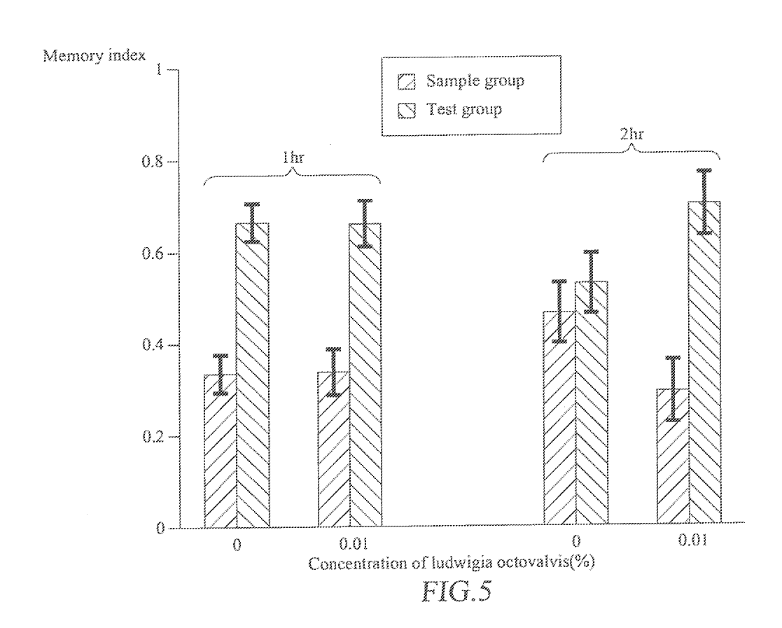

FIG. 5 shows a graph of an experiment carried out in the present invention illustrating the concentration of an extracted Ludwigia octovalvis substance relative to memory performance in mice using novel object recognition task. The mice used herein, are 3 months old senescence-accelerated mouse prone-8 (SAMP8). It has been shown that SAMP8 have a shorter lifespan and develop Alzheimer's disease like phenotype around 6-12 month of age. In this experiment, 0 or 0.0.1% of the extracted Ludwigia octovalvis substance are added into the drinking water of 3 months old SAMP8 mice and the memory performance of these mice are tested after 3 months.

SMAP8 mice treated with 0 or 0.01% of the extracted Ludwigia octovalvis substance are placed in an box (20×20×10 inches). During the training period, two different training objects are placed within the box, and mice are allowed to explore the two training objects for 5 minutes. After a period of time (1 hr or 2 hrs), one of the training objects is replaced by a new object and the mice are allowed to freely explore for another 5 minutes. The time mice spend on new object, and the old object are recorded. The object recognition index is compiled by dividing the spent time for exploring the old object, or a new object with the total time for exploring the old object and the new object. The result achieved thereby is multiplied by 100%.

FIG. 5 shows when the memory test carried out one hour after the training period, the mice treated with 0% and 0.01% of the extracted Ludwigia octovalvis substance both spent more time on exploring the new object than on the old object, suggesting that both groups of mice can distinguish between the training object and the new object. However, only mice had treated with 0.01% of the extracted Ludwigia octovalvis substance can still discriminate the training object and the new object when memory test are carried out 2 hrs after the training period. In summary, the experiment described above proves that the extracted Ludwigia octovalvis substance can effectively enhance the memory performance in SAMP8 mice.

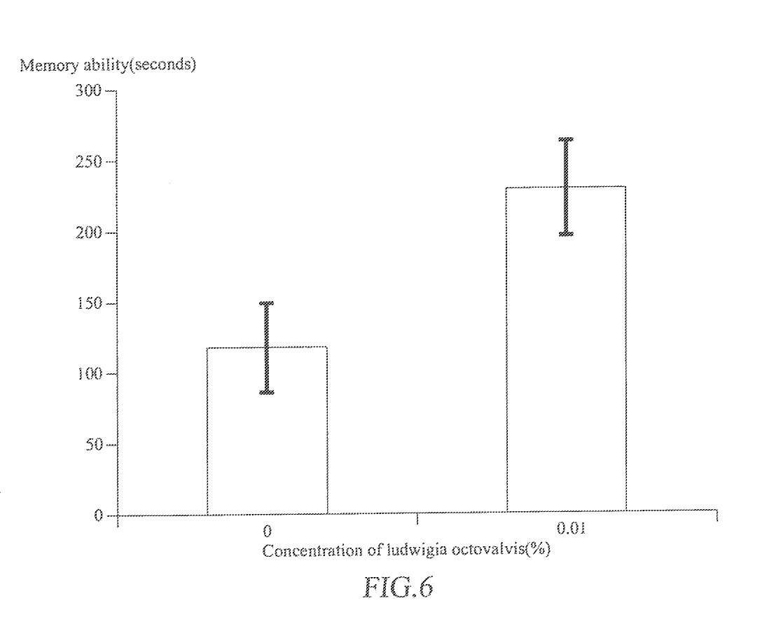

FIG. 6 shows a graph of an experiment carried out in the present invention, illustrating the concentration of an extracted Ludwigia octovalvis substance relative to memory performance In SAMP8 mice using elevated T-maze task. The elevated T-maze is made of plastic and has three arms of equal dimensions (30 cm×6 cm). One arm is enclosed by walls of 16 cm high and is perpendicular to two opposing open arms. The whole apparatus was elevated 40 cm above the floor.

SMAP8 mice are bred and treated with 0 and 0.01% of the extracted Ludwigia octovalvis substance as described in FIG. 5. On the training day within the elevated T-maze task, each mouse is placed at the distal end of the enclosed arm facing the intersection of the arms and is allowed to explore the enclosed arm. The trial ended when the mouse entered one of the open arms by placing all four paws into the open arm or remained in the enclosed arm for a maximum of 300 seconds. In the training session, mice were re-exposed to the elevated T-maze as many times as needed to remain in the enclosed arm for 300 seconds. 24 hours after the training, mice were re-exposed to the enclosed arm and the time that the animal remained in the enclosed arm was recorded and used to assess memory performance.

From the above experiment, 24 hours after the training, mice treated with 0.01% of the extracted Ludwigia octovalvis substance show better memory performance on the elevated T-maze task, as they remain in the closed space for a longer period of time compared to mice treated with 0% of the extracted Ludwigia octovalvis substance.

For those persons having general knowledge in the technical field, by reading this invention should understand the following features:

(i) Since the Ludwigia octovalvis is dipped in the extraction solution at low temperature, the extraction solution soaks through the Ludwigia octovalvis in such a manner to extract a Ludwigia octovalvis extraction solution and a Ludwigia octovalvis residue. In addition, the Ludwigia octovalvis is ground prior to the dipping operation, extraction of Ludwigia octovalvis substance is effective, and heating of the extraction solution facilitates and enhances extraction of the Ludwigia octovalvis substance.

(ii) Since a drying process is conducted to the Ludwigia octovalvis extraction solution, the extracted Ludwigia octovalvis substance thus can be obtained at a higher concentration, the drugs produced accordingly enhance the brain functions and delay neurodegneration.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A method for manufacturing drugs that enhance brain function, wherein Ludwigia octovalvis is used as raw material, the method comprising steps of:
(a) dip a predetermined amount of Ludwigia octovalvis in a first extraction solution to extract a first Ludwigia octovalvis extraction solution and a Ludwigia octovalvis residue;
(b) separate the Ludwigia octovalvis residue from the first Ludwigia octovalvis extraction solution; and
(c) conduct a drying process to the first Ludwigia octovalvis extraction solution to obtain an extracted Ludwigia octovalvis substance.

2. The method according to claim 1, further comprising after the (c), a step (d), where the extracted Ludwigia octovalvis substance is redissolved in the first Ludwigia octovalvis extraction solution in such a manner that the extracted Ludwigia octovalvis substance has a concentration ranging between 0.0005%˜0.5%

3. The method according to claim 1, wherein in the step (a), said predetermined amount of Ludwigia octovalvis is dipped in the first extraction solution for 4˜12 hours.

4. The method according to claim 3, wherein in the step (a), said predetermined amount of Ludwigia octovalvis is dipped in the first extraction solution at a low temperature ranging 2˜10° C.

5. The method according to claim 3, wherein in the step (a), after said predetermined amount of Ludwigia octovalvis is dipped in the first extraction solution for 4˜12 hours, after which the first extraction solution is heated for 10˜60 minutes.

6. The method according to claim 1, wherein the (b) further including a substep (b1): where the Ludwigia octovalvis residue is dipped in a second extraction solution, after which the second extraction solution is heated and filtered in such a manner to extract a second Ludwigia octovalvis extraction solution, which, in turn, is combined in the first Ludwigia octovalvis extraction solution.

7. The method according to claim 1, wherein in the step (a): said predetermined amount of Ludwigia octovalvis is ground prior to dipping said predetermined amount of Ludwigia octovalvis in the first extraction solution.

8. The method according to claim 1, wherein in the step (b): separating the Ludwigia octovalvis residue from the first Ludwigia octovalvis extraction solution is conducted via centrifugal filtering means.

9. The method according to claim 1, wherein the first extraction solution is water-solvent.

10. The method according to claim 9, wherein in the step (a), after said predetermined amount of Ludwigia octovalvis is dipped in the first extraction solution, the first extraction solution is heated at a temperature ranging from 50° C.˜100° C.

11. The method according to claim 9, wherein the drying process in the (c) is a freeze drying process.

12. The method according to claim 1, wherein the first extraction solution is an alcohol solution.

13. The method according to claim 12, wherein in the step (a), after dipping said predetermined amount of Ludwigia octovalvis in the first extraction solution, the first extraction solution is heated indirectly through a wafer bath up to a temperature ranging from 50° C.˜100° C.

14. The method according to claim 12, wherein said drying process in the step (c) is a decompress concentration process.
